SEQUENCE LISTING (18 sequences)

SEQ ID NO: 1 - DNA, 1578 bp, Rosa sp.
  Nucleotide sequence of gene encoding rose flavonoid glycosyl transferase
gctggagctctatcaatctccccttaaagtcgttggtcgatcctcttcatttcagttcatttctcacttgaaaggctgaaagtttgcacaatgagccacaaacttgctagttctaatcctcaagcccctgtggcagtttttaattttccattcgcctcccatcccagctctcttctccgatttatacgaaccatttcggccgttgccccggacattaagttcaccttcttcagcacttctagaacaaatagctccctcttctcgggttccaacaacaagggcttggataatgtaaagccttacaatgtatgggatggcttgccggagggttacgtgcccccctctggacctccgctggagcaaattgggttgttccttgaccaggcacctcaaagctttgagagggcactcaaagaagtggaggccgaaacaggacacaagttcggctgcttgatttcggatgcatttctctggttttccggggacattgcccagaaaatgaacgtcccttgggtgactacatggagtggaccacgcccgctgcttgttcatctggagactgatatgatcagagaaaaagtgggagctcctgggcaagaagacaagaccctggacttccttccaggattctcaaatgagttcctagcatctgacttaccaaaggaagttgtatttggaaacatagagtcgccacttgcaaatatgttgtataaaatgggacagaagctgccacaagcaactgcggttgcagtcaactcttttgaaacaatggacttgaaagtttccgaggagctgaagaaaagactcaagaagttgcttcttgttgggccactacatctagtccggccagtaccatcagcacaatcagatgatgacgaggaggagaaggatgcatgcttaccgtggctggacaaccacaagcctgcatcagtagcatacatcagctttggaagtgtcggagcactgcccccaatggaggtagcagcattagccgaggcattagaggaagggggattcccatttctttggtcatttagggggaacctagaggactttcccaaaggattcattgaaagaacatctactggaagagttgttccctgggtgaaccaggtgcaaatcctaaatcatccgtcgataggggtgtttgttacacatggcggttggaagtcagttttggagagtgtaacttgtggtgtgcctatgattgggaggcctcattttgctgatcaaactctgaatatgcggagcgtagaagttgtatggaagatcggtatgagaatcgagggtggcgttttcactaaatctggagcaatcaaggtattggaacaagctctatcgcttgagcaaggaaaagaaatgagacagagagttggagtccttaaacagcttgctcaagagactgttggacccaatgggagttcagctcaagacttgaaagtctggtagagatcatcaaatcatgattgcgaagtttaattactgttggtctgaaatcatgtcatgatggccaatgcatctgagtttaaatcatctatcaataatttctgttctggtcttaaaaaaaaaaaaaaaaaa

SEQ ID NO: 2 - protein, 452 aa, Rosa sp.
  Amino acid sequence of rose flavonoid glycosyl transferase
MSHKLASSNPQAPVAVFNFPFASHPSSLLRFIRTISAVAPDIKFTFFSTSRTNSSLFSGSNNKGLDNVKPYNVWDGLPEGYVPPSGPPLEQIGLFLDQAPQSFERALKEVEAETGHKFGCLISDAFLWFSGDIAQKMNVPWVTTWSGPRPLLVHLETDMIREKVGAPGQEDKTLDFLPGFSNEFLASDLPKEVVFGNIESPLANMLYKMGQKLPQATAVAVNSFETMDLKVSEELKKRLKKLLLVGPLHLVRPVPSAQSDDDEEEKDACLPWLDNHKPASVAYISFGSVGALPPMEVAALAEALEEGGFPFLWSFRGNLEDFPKGFIERTSTGRVVPWVNQVQILNHPSIGVFVTHGGWKSVLESVTCGVPMIGRPHFADQTLNMRSVEVVWKIGMRIEGGVFTKSGAIKVLEQALSLEQGKEMRQRVGVLKQLAQETVGPNGSSAQDLKVW

SEQ ID NO: 3 - DNA, 3507 bp, Rosa sp.
  Nucleotide sequence of gene encoding rose flavonoid glycosyl transferase
aagcttgacccatgacgacgacgacggcaatttcacttcaaaaaaaaaagaaaaaaaagaaagaaaggaaaacagagaaaaagaaaaggtcgccgatctctttctctgcttgtataaagtgggtgatggactttaggagttggggggcgactaaattagcagacttgcggtggcgatcaatttttagagagataaacaacccgcgccagccgtgaggagatttatgagtgatagagagaaaatagaaacaaagtaaaggtcgcgtttcttttctaatttctgcagcaacccaaaaagaataacacatgagatattctttcattttctttcttttagtggacttactgacccagctttcaggcccatcttgggcctattgggctttttcttgaaatgagtcaacctgtcttaaaccaatctggctcgtgggcctggccgggggttaccgcgtgctcaagtgttactgtagaagcctgccggtctcctcctagaaataaaacttttgtttagtggttggcttgtgacaaccaccgttgttactatactactttggttctataaatacagaccaaagccacatggaaaaaatagaattgcgaccactgaagtaggtccatatatatatatccctcacgtaacttgagctttacgtagtttctatacccaaaccttgacttcatggcaccagcatcaaatcaggtgggtggtcatgtggcggtgctagcctttcccttctcgactcacgccgccccgctgttgaacatcgtctgccgcctcgccgccgccgccccaaacaccctcttctcattcttcaacaccaagcaatccaatagctcgatcctagcaagcaacacgtcatccatactacgtaacagtaacgtaagggtgtgtgaggtggcggatggtgtgccggaggggtatgtttttgtgggtaagcctcaggaggacatagagttgttcatgaaggctgccccggacaacttcaggcggtgcttagaggcgtccgtggcggagaccgggagggaggtcagctgcttggtcactgacgctttcttttggttcggggctcacatggccgatgacttgggaggactgccttgggtaccgttttggaccgccgggccagcttcactctccgctcatgtacacaccgatctcatcaggaacacaactagtacgtgtatacatatattatgattcatcaatatcatatataccgaattattaatgctttatttaattagtctcgtcatgtgtgtaccaaaacaagcagacacaagtacacatacatatctattgacaccaaaatggttatattactagctgacttgtctcgatcattcatatgttcaaataaacaggtatgggtggtcacgatggaaaagaaaccatcactgcagtcactgcaggaatgtccaaagtacgaccccaggatttgccagagggaatcatctttggaaagttggactccctcttttcacggatgctacaccagatgggactaatgctaccacttgcaacagcagttttcatcaactccttcgaagaactagaccctgtgatcacaaatgatttgaagtccaaattcaagaggtacctcaacgtgggacccttcgacctactagagtcaccagcaccagcagccaccaccacactgcagactggggacgttgttgtcggagatggctgcttatcgtggcttgacaaacagaaggcggcgtctgtagtgtatgtgagttttggatcagtaacaagaccatcgccggaggagcttatggcgctagccgaggcgttggaagccagtagggttccattcttgtggtctcttcggaacaatttgatgactcctaagctagacgagttcataagcaaagcagaattaaacgggatggtggtgccttgggtgccacaaccacaggtcctagcacatggttcagttggagcattcgtaacacattgtggttggaactcggtgcttgagagcctagcaggtggagtgcccatgatctgcaggcctttcttcggcgatcagaaacttaacgcgaggatggtagaggatgagtggaagattggtctcaaattggagggtggggttttcaccaagaatgggatgcttaagagtttggacatactactgtcgcaaaagaaggggaacataatgagagacacgatcaacacattcaaacaactcgcacaacaggccgtagaaccaaaagggagctccactaggaactttgaatcattgttggaagtgatatctacagccaattaagtataaccacaaatatattccagaagaacaaagaactactaagggttacagttgaatgaaagtgcttcgacttgtctgaataaaattctacttatacttctaatcatccttcatagaaaagccatagttcttcatacaaccttgctcaaatatgctagtcgtcttgttgatagtacaaaatatcatcgtttacactgaaactaccgtatccaacaaaaatcaagcaaactattcactaacatagaggactatatatattggacccaatcgaattgaatctgtgggaggaaagaaaaccaaagcattgagttgccccaataagaggggcaagatatttctgcacaacccataaaggtaagtatacattacactaagaagaattatcaatattaatagtcatttaaacagattgaatcccatctagcaagtacatatagatcaatctggatccagcaaaacccgtacaaatgactcgacatacccctctccctaaatcagcacctaaatactctacagccatccaatagacatataggcagtgcagccagcgggctacgggtgcccttgtgcttttactaattttgaccaagaaaattctggaaacaaaagggaatctaattagggaaaaaatgctgtatttgcattgacagattccaacaactgaatctaggtaccgcactgcatctccatcaatcacccatgaatttagctcccacatgaatattagccatctaggaatcaaccatttcatcttgtatctcattgggggcaacgagcccaggaatagaaagcatccgttgccgttctctttctctttgtacagcagcctcctctgcataaagatctttgttgtcctgcaacagagaaagcaaacctctaattatacttgactaaaatcacaatgtctgacaaagtaacagtgacttatcccaagcataaaacagattcaagaaaaagaacatcatttgatactggtagtatcctacctgtgctgaaaattcttttgattgcactagaaagtctcggatgtgatccttaaatgtggccaagtcattcttggactcgaatagtccattcacaaattgagtgacctgaggaaaaaaaaaaaaacaaataaataaaacccttcagatgagtggtattcttatggaattagtattctgggtacaaacaaaataatattagaaatatacctctatggaagtcatgttggggaaggaatcactcaaaagctt

SEQ ID NO: 4 - protein, 468 aa, Rosa sp.
  Amino acid sequence of rose flavonoid glycosyl transferase
MAPASNQVGGHVAVLAFPFSTHAAPLLNIVCRLAAAAPNTLFSFFNTKQSNSSILASNTSSILRNSNVRVCEVADGVPEGYVFVGKPQEDIELFMKAAPDNFRRCLEASVAETGREVSCLVTDAFFWFGAHMADDLGGLPWVPFWTAGPASLSAHVHTDLIRNTTGMGGHDGKETITAVTAGMSKVRPQDLPEGIIFGKLDSLFSRMLHQMGLMLPLATAVFINSFEELDPVITNDLKSKFKRYLNVGPFDLLESPAPAATTTLQTGDVVVGDGCLSWLDKQKAASVVYVSFGSVTRPSPEELMALAEALEASRVPFLWSLRNNLMTPKLDEFISKAELNGMVVPWVPQPQVLAHGSVGAFVTHCGWNSVLESLAGGVPMICRPFFGDQKLNARMVEDEWKIGLKLEGGVFTKNGMLKSLDILLSQKKGNIMRDTINTFKQLAQQAVEPKGSSTRNFESLLEVISTAN

SEQ ID NO: 5 - DNA, 21 bp, Artificial Sequence
  Description of Artificial Sequence Synthetic probe
cacattggcgttctagcattc

SEQ ID NO: 6 - DNA, 21 bp, Artificial Sequence
  Description of Artificial Sequence Synthetic probe
gccttcgggggtcccgtccca

SEQ ID NO: 7 - DNA, 21 bp, Artificial Sequence
  Description of Artificial Sequence Synthetic probe
catgttgcagtgcttgcattt

SEQ ID NO: 8 - DNA, 21 bp, Artificial Sequence
  Description of Artificial Sequence Synthetic probe
tccctccggcgagccatccca

SEQ ID NO: 9 - DNA, 21 bp, Artificial Sequence
  Description of Artificial Sequence Synthetic probe
cacattggcgtgctagcgttc

SEQ ID NO: 10 - DNA, 21 bp, Artificial Sequence
  Description of Artificial Sequence Synthetic probe
gccctctggggtgccatccca

SEQ ID NO: 11 - DNA, 21 bp, Artificial Sequence
  Description of Artificial Sequence Synthetic probe
cacattgcacttcttgctttc

SEQ ID NO: 12 - DNA, 21 bp, Artificial Sequence
  Description of Artificial Sequence Synthetic probe
tgtttcagtgacaccatccca

SEQ ID NO: 13 - DNA, 25 bp, Artificial Sequence
  Description of Artificial Sequence Synthetic primer
gactccatggcaccagcatcaaatc

SEQ ID NO: 14 - DNA, 25 bp, Artificial Sequence
  Description of Artificial Sequence Synthetic Rose3GT16-Spe-I-1 primer
acacgtactagttgtgttcctgatg

SEQ ID NO: 15 - DNA, 29 bp, Artificial Sequence
  Description of Artificial Sequence Synthetic Rose3GT16-SpeI-3 primer
agtcactagtatgggtggtcacgacgatg

SEQ ID NO: 16 - DNA, 24 bp, Artificial Sequence
  Description of Artificial Sequence Synthetic Rose3GT16-KpnI primer
cgttgaggtacctcttgaatttgg

SEQ ID NO: 17 - DNA, 22 bp, Artificial Sequence
  Description of Artificial Sequence Synthetic pSPB1123-SphI primer
agagttgcatgcaaccgcagtt

SEQ ID NO: 18 - DNA, 16 bp, Artificial Sequence
  Description of Artificial Sequence Synthetic reverse primer
aacagctatgaccatg

(19) United States
(12) Patent Application Publication
(10) Pub. No.: US 2010/0306877 A1
(43) Pub. Date: Dec. 2, 2010

(54) FLAVONOID GLUCOSYLTRANSFERASE AND USE OF SAME

(75) Inventors: Yoshikazu Tanaka, Osaka (JP); Yukihisa Katsumoto, Osaka (JP); Yuko Fukui, Osaka (JP); Masako Mizutani, Osaka (JP); Junichi Togami, Osaka (JP)

Correspondence Address: DRINKER BIDDLE & REATH (DC), 1500 K STREET, N.W., SUITE 1100, WASHINGTON, DC, 20005-1209, US

(73) Assignee: International Flower Development Proprietary Limited, Victoria (AU)

(21) Appl. No.: 12/279,708

(22) PCT Filed: Feb. 16, 2007
(86) PCT No.: PCT/JP2007/053342

§ 371 Date: Aug. 15, 2008

(30) Foreign Application Priority Data
Feb. 17, 2006 (JP) 2006-041280

(51) Int. Cl. A01H 5/00; C12N 15/82; C07H 21/04; C12N 9/10

(52) U.S. Cl. 800/282; 800/298; 536/23.2; 435/320.1; 435/193

(57) ABSTRACT

A novel flavonoid glucosyltransferase originating from rose, a nucleic acid encoding that enzyme, for example, a flavonoid glucosyltransferase having an amino acid sequence indicated in SEQ ID NO. 2 or SEQ ID NO. 4, a nucleic acid encoding that enzyme, a method for changing the color of a rose flower using that nucleic acid, and a rose plant capable of changing color, are provided.

### TECHNICAL FIELD

The present invention relates to the gene of an enzyme that glycosylates flavonoids and a method for using the same.

### BACKGROUND ART

Since flowers exhibiting new traits are always considered to have significant value in the ornamental plant industry, the development of such ornamental plants is industrially important. Flower color is the most important trait of ornamental plants. Cultivars with various colors have been bred by breeding mainly with using of hybridization breeding. However, in the case of breeding by hybridization breeding, since available genetic resources are limited to closely related plants that can be hybridized, there are many cases in which plant color is limited, and it is rare that a single plant species has cultivars exhibiting all colors. For example, blue roses and carnations, yellow morning glories and geraniums etc. were unable to be developed by breeding with using hybridization breeding.

The main components of flower color are a group of flavonoid compounds generically referred to as anthocyanins. A wide range of anthocyanins are known to exist in plants, and many of their structures have already been determined. The color of anthocyanins is primarily dependent upon their structures. Anthocyanins known to be contained in roses are cyanidin 3,5-diglucoside, pelargonidin 3,5-diglucoside, cyanidin 3-glucoside, pelargonidin 3-glucoside, peonidin 3,5-diglucoside and peonidin 3-glucoside. These determine the colors of orange to red in rose flowers. A blue compound generically referred to as rosacyanin is contained in some roses, and rosacyanin is a derivative of cyanidin, which is a type of anthocyanidin (Tetrahedron Letters (2002) 43, 2637-2639, Japanese Unexamined Patent Publication No. 2002-201372, Tetrahedron (2006) 62, 9661-9670).

Considerable research has been conducted on enzymes and genes involved in the biosynthesis of anthocyanins (Plant Cell, Tissue and Organ Culture (2005) 80, 1-24). There are some examples that the structure of anthocyanins are modified to change flower color by introducing and expressing these genes in plants (Plant Cell, Tissue and Organ Culture (2005) 80, 1-24). The biosynthesis of anthocyanins leading to anthocyanidin 3-glucosides is conserved among nearly all flowering plants, and anthocyanidins are typically first modified with a sugar such as glucose at the 3 position (Plant Cell, Tissue and Organ Culture (2005) 80, 1-24). Alternatively, galactose is added instead of glucose depending on the plant species, resulting in the accumulation of anthocyanidin 3-galactosides.

However, in roses, which are industrially important horticultural plants, anthocyanidin 3-glucosyltransferase activity has not been detected, while, differing from other flowering plants, anthocyanidin 5,3-glucosyltransferase (A5,3GT) is present. Namely, a glucose is reported to transfer to the 3 position of an anthocyanidin after having transferred to the 5 position (Nature (2005) 435, 757-758). On the other hand, the blue rosacyanin present in some roses shows structure containing cyanidin. Although it is not known how this is biosynthesized from cyanidin, based on the structure of rosacyanin, it is clear that the reaction in which a sugar is transferred to cyanidin is unnecessary for the biosynthesis of rosacyanin.

R═H(A1) or

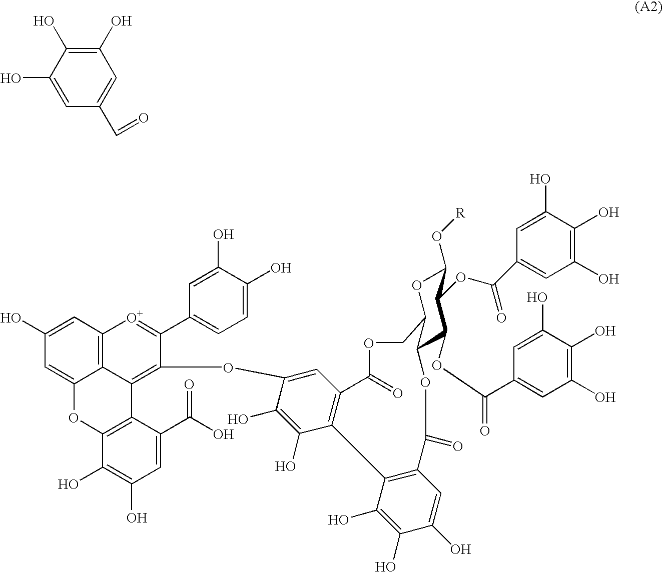

On the basis of the above, it is suggested that the presence of glucosyltransferase activity toward anthocyanidins is important for determining whether anthocyanin or rosacyanin is biosynthesized in rose petals, namely if anthocyanidin glucosyltransferase activity is present, anthocyanin is synthesized, while if anthocyanidin glucosyltransferase activity is not present, rosacyanin is synthesized. On the basis of previous findings (Nature (2005) 435, 757-758), in the case of roses, it has been suggested that the anthocyanidin glucosyltransferase thereof is not anthocyanidin 3-glucosyltransferase, but rather anthocyanidin 5,3-glucosyltransferase (A5,3GT).

Enzymes that transfer sugars to various compounds including flavonoids use sugar donors such as UDP-glucose or UDP-rhamnose and catalyze reactions that transfer a sugar to a hydroxyl group to a specific or non-specific location of a compound. Such glucosyltransferases form a glucosyltransferase family in the plant genome, and more than 100 kinds of molecular species are known to exist for a single species (Glycobiology (2003) 13, 139-145).

Glucosyltransferases, which transfer sugars to flavonoids, including anthocyanidins, also belong to this glucosyltransferase family. There are a comparatively large number of findings describing enzymes that transfer sugars to flavonoids within this family. In many cases, enzymes that transfer sugars to flavonoids catalyze a reaction that transfers a sugar to a hydroxyl group at a specific position on the flavonoid, and the amino acid sequences of enzymes that transfer sugars to the same position are similar irrespective of plant species.

Reactions that transfer a sugar to the 3 position or the 5 position of flavonoids, including anthocyanins, have thus far been known to be catalyzed by different enzymes such as flavonoid (or anthocyanidin) 3-glucosyltransferase (A3GT) and anthocyanin 5-glucosyltransferase (A5GT), respectively, and genes that encode these enzymes have been obtained from several plants. In addition, each of these enzymes respectively form sub-families (Plant Mol. Biol. (2002) 48, 401-11, Current Opinions in Plant Biology (2005) 8, 1-10). In addition, the amino acid sequences of enzymes transferring sugars to the 3′ position and the 7 position of flavonoids exhibit a comparatively high degree of homology, and flavonoid 7-glucosyltransferase, which transfers sugar to the 7 position of flavonoids (Planta. (2000) 210, 1006-1013) and gentian anthocyanin 3′-glucosyltransferase, which transfers sugar to the 3′ position of anthocyanins (Plant Physiol. (2003) 132, 1652-1663), belong to the same subfamily. On the other hand, rose A5,3GT belongs to a different sub-family.

By the way, some cultivars of roses are known to change color after they have bloomed, examples of which include cultivars such as Masquerade and EHIGASA. Although these cultivars have a yellow to cream color from bud to the early stages of blooming, during blooming progresses, flower color changes to red or pink. In these color-changing roses, anthocyanins are synthesized in the bud stage similar to other rose cultivars. However, since their flower colors change to a red or pink color, there is believed to probably be a stage during which anthocyanins are again actively synthesized even after blooming. The Masquerade cultivar produced in 1949 is said to be the first cultivar to change color after blooming (Encyclopedia of Roses, Japan Broadcast Publishing Co., Ltd., ISBN 4-14-645776-9). Such traits are inherited in subsequent generations by hybridization breeding and although new cultivars have been developed, coloring following blooming is limited to red or pink.

In addition, A3GT gene has recently been reported to be expressed accompanying anthocyanin synthesis in cultured cells of a similarly color-changing rose cultivar known as Charleston (Plant Biotechnology (2006) 23, 379-385).

On the basis of the current level of technology, it is possible to produce a genetically recombinant rose where an exogenous gene is overexpressed or an endogenous gene is inhibited therein.

Patent Document 1: Japanese Unexamined Patent Publication No. 2002-201372

Non-Patent Document 1: Tetrahedron Letters (2002) 43, 2637-2639

Non-Patent Document 2: Plant Cell, Tissue and Organ Culture (2005) 80, 1-24

Non-Patent Document 3: Nature (2005) 435, 757-758

Non-Patent Document 4: Glycobiology (2003) 13, 139-145

Non-Patent Document 5: Plant Mol. Biol. (2002) 48, 401-11

Non-Patent Document 6: Planta. (2000) 210, 1006-1013

Non-Patent Document 7: Plant J. (2003) 132, 1652-1663

Non-Patent Document 8: Tetrahedron (2006) 62, 9661-9670

Non-Patent Document 9: Plant Biotechnology (2006) 23, 379-385

### DISCLOSURE OF THE INVENTION

An enzyme, and gene thereof, which transfers a sugar to the 3 position (before transferring to the 5 position) of anthocyanins in roses, or an enzyme, and gene thereof, which transfers a sugar only or preferentially to the 3 position, are not known, and such enzymes are not considered to exist (Nature (2005) 435, 757-758). If such enzymes or genes were able to be obtained, it would be possible to alter the biosynthesis pathway of anthocyanin and rosacyanin in roses, thereby clearly making it possible to alter the flower color of roses. In addition, as is observed in roses that change color accompanying blooming, if it were possible to acquire a gene involved in anthocyanin synthesis involved in color change after blooming (including expression regulating moieties such as the promoter of that gene), it would be possible to develop a rose that changes color after blooming by molecular breeding using that gene.

Thus, the present invention provides a nucleic acid encoding a protein originating in family Rosaceae genus rosa having activity that transfers a sugar to a 3-position hydroxyl group of a flavonoid. In particular, the flavonoid is anthocyanidin or flavonol.

In addition, the present invention provides a nucleic acid encoding a protein having an amino sequence indicated in SEQ ID NO. 2 or SEQ ID NO. 4, which has activity that transfers a sugar to a 3-position hydroxyl group of a flavonoid.

Moreover, the present invention provides a nucleic acid encoding a protein having an amino acid sequence that has been modified by addition, deletion and/or substitution with other amino acids to one or a plurality of amino acids in an amino acid sequence indicated in SEQ ID NO. 2 or SEQ ID NO. 4, and having activity that transfers a sugar to a 3-position hydroxyl group of a flavonoid, and more specifically, provides a nucleic acid encoding a protein having an amino acid sequence that has been modified by addition, deletion and/or substitution with other amino acids to one or several amino acids in an amino acid sequence indicated in SEQ ID NO. 2 or SEQ ID NO. 4, and having activity that transfers a sugar to a 3-position hydroxyl group of a flavonoid.

In addition, the present invention provides a nucleic acid encoding a protein having sequence identity of at least 60% with respect to an amino acid sequence indicated in SEQ ID NO. 2 or SEQ ID NO. 4 and having activity that transfers a sugar to a 3-position hydroxyl group of a flavonoid; for example, a nucleic acid encoding a protein having sequence identity of at least 80% with respect to an amino acid sequence indicated in SEQ ID NO. 2 or SEQ ID NO. 4 and having activity that transfers a sugar to a 3-position hydroxyl group of a flavonoid; and more specifically, a nucleic acid encoding a protein having sequence identity of at least 90% with respect to an amino acid sequence indicated in SEQ ID NO. 2 or SEQ ID NO. 4 and having activity that transfers a sugar to a 3-position hydroxyl group of a flavonoid.

Moreover, the present invention provides a nucleic acid encoding a protein having an amino acid sequence encoded by a nucleic acid that hybridizes under moderate stringent conditions with a nucleic acid having a nucleotide sequence of a coding region indicated in SEQ ID NO. 1 or SEQ ID NO. 3, and having activity that transfers a sugar to a 3-position hydroxyl group of a flavonoid; and more specifically, provides a nucleic acid encoding a protein having an amino acid sequence encoded by a nucleic acid that hybridizes under high stringent conditions with a nucleic acid having a nucleotide sequence of a coding region indicated in SEQ ID NO. 1 or SEQ ID NO. 3, and having activity that transfers a sugar to a 3-position hydroxyl group of a flavonoid.

A typical gene of the present invention is a nucleic acid containing a nucleotide sequence of a coding region indicated in SEQ ID NO. 1 or SEQ ID NO. 3.

A nucleic acid of the present invention is DNA or RNA, and particularly DNA. Moreover, the present invention provides a vector containing this DNA and a host that has been transformed by this vector.

Moreover, the present invention provides a plant into which any of the nucleic acids described above has been inserted, a progeny thereof having identical properties thereto, or tissues or vegetatively propagated plants thereof; a plant in which any of the nucleic acids described above has been inserted and for which flower color has been altered, progeny thereof having identical properties thereto, or tissue or vegetative growth body thereof; and, a cut flower of the above plants or progeny thereof having identical properties thereto.

Moreover, the present invention provides a protein encoded by any of the various nucleic acids described above. In addition, the present invention provides a method of producing a protein having activity that transfers a sugar to a 3-position hydroxyl group of a flavonoid, by culturing or growing the host, and then harvesting the protein from the host.

### BRIEF DESCRIPTION OF THE DRAWINGS

FIG. 1 shows the expression of F3GT gene and A3GT gene in the petals of buds of various cultivars of roses. The figure is resulted from Northern hybridization of RNA obtained from the petals of buds in which anthocyanin synthesis is active using a DIG RNA probe originating in the F3GT sequence or A3GT sequence.

FIG. 2 shows the state of petals at each developmental stage of a rose (cultivar: Ehigasa) (when flower color is changing from orange-yellow to red) (A), and the amount of anthocyanin present in the petals at each growth stage (B).

FIG. 3 shows the expression of A3GT gene and A5,3GT gene in petals at each developmental stage of a rose (cultivar: Ehigasa). Expression of A3GT gene, A5,3GT gene and GAPDH gene at each growth stage were investigated by Northern hybridization. DIG-labeled RNA probes originating in each gene sequence were used. The flower growth stages of 1 to 4 correspond to growth stages 1 to 4 in FIG. 2.

FIG. 4 shows the expression of CHS gene, F3′H gene, DFR gene and A3GT gene in petals at each developmental stage of a rose (cultivaer: Ehigasa). Expression of CHS gene, F3′H gene, DFR gene and A3GT gene at each developmental stage was investigated by Northern hybridization. DIG-labeled RNA probes originating in each gene sequence were used. The flower growth stages of 1 to 4 correspond to growth stages 1 to 4 in FIG. 2.

FIG. 5 shows changes in flower color in the case of introduction of pSFL224, which controls expression of F3GT, into a rose host (cultivar: Lavande). The reddish-violet color of the host became considerably lighter in the transformants.

FIG. 6 shows the expression of F3GT gene and A5,3GT gene in a rose host (cultivar: Lavande) and an F3GT-inhibited transformant (same as FIG. 5). Expression of F3GT gene, A5,3GT gene and GAPDH gene in the Lavande host and F3GT-inhibited transformant was investigated by Northern hybridization. DIG-labeled RNA probes originating in each gene sequence were used. A portion present only in endogenous F3GT gene was used for the F3GT 600 by probe. The flower developmental stages of 1 to 4 correspond to developmental stages 1 to 4 in FIG. 2.

### BEST MODE FOR CARRYING OUT THE INVENTION

A protein encoded by the nucleic acid of the present invention having activity that transfers a sugar to a 3-position hydroxyl group of a flavonoid most typically has an amino acid sequence indicated in SEQ ID NO. 2 or SEQ ID NO. 4. However, it is known in the technical field of enzymes that an essential region involved in enzyme activity or substrate specificity and the like and a non-essential region for enzyme activity and other properties are present within the enzyme protein, and the enzyme activity and other important properties are maintained even if the amino acid sequence in the non-essential region is modified by addition or deletion of amino acids or substitution of amino acids with other amino acids.

Thus, the present invention provides not only a nucleic acid encoding an enzyme having an amino acid sequence indicated in SEQ ID NO. 2 or SEQ ID NO. 4, but also provides a protein having an amino acid sequence that has been modified by addition, deletion and/or substitution with other amino acids to one or a plurality of amino acids in an amino acid sequence indicated in SEQ ID NO. 2 or SEQ ID NO. 4, and having activity that transfers a sugar to a 3-position hydroxyl group of a flavonoid, as well as a nucleic acid encoding that protein. For example, the present invention provides a protein having an amino acid sequence that has been modified by addition, deletion and/or substituted with other amino acids to one or several amino acids in an amino acid sequence indicated in SEQ ID NO. 2 or SEQ ID NO. 4, and having activity that transfers a sugar to a 3-position hydroxyl group of a flavonoid, and a nucleic acid encoding that protein. The number of amino acids subjected to modification is 50 or less, for example 40 or less, 30 or less or 20 or less, and typically 10 or less.

Enzyme activity is maintained even if modifications are added to a naturally-occurring amino acid sequence as was previously described. As will be described later, the amino acid sequence of the enzyme of the present invention only has amino acid sequence identity of less than 60% with an amino acid sequence having the highest sequence identity of a known enzyme having similar activity. Thus, the present invention provides a protein having sequence identity of at least 60% with respect to an amino acid sequence indicated in SEQ ID NO. 2 or SEQ ID NO. 4 and having activity that transfers a sugar to a 3-position hydroxyl group of a flavonoid, and a nucleic acid encoding that protein; a protein having sequence identity of at least 80% with respect to an amino acid sequence indicated in SEQ ID NO. 2 or SEQ ID NO. 4 and having activity that transfers a sugar to a 3-position hydroxyl group of a flavonoid, and a nucleic acid encoding that protein; and more limitedly, a protein having sequence identity of at least 90% with respect to an amino acid sequence indicated in SEQ ID NO. 2 or SEQ ID NO. 4 and having activity that transfers a sugar to a 3-position hydroxyl group of a flavonoid, and a nucleic acid encoding that protein.

It is known that if a gene encoding a certain enzyme is cloned, a gene encoding a different enzyme having similar activity can be cloned by using a probe and primers designed on the basis of the sequence data of that gene. Thus, the present invention also provides a protein having an amino acid sequence encoded by a nucleic acid that hybridizes under moderate stringent conditions with respect to a nucleic acid having a base sequence of a coding region indicated in SEQ ID NO. 1 or SEQ ID NO. 3 and having activity that transfers a sugar to a 3-position hydroxy group of a flavonoid, and a nucleic acid encoding that protein; and more specifically, a protein having an amino acid sequence encoded by a nucleic acid that hybridizes under high stringent conditions with respect to a nucleic acid having a base sequence of a coding region indicated in SEQ ID NO. 1 or SEQ ID NO. 3 and having activity that transfers a sugar to a 3-position hydroxy group of a flavonoid, and a nucleic acid encoding that protein.

The aforementioned moderate stringent conditions refer to hybridizing overnight at 37° C. in 5×SSC containing 30% formaldehyde followed by washing at 55° C. in 5×SSC containing 1% SDS, while the high stringent conditions refer to hybridizing overnight at 42° C. in 5×SSC containing 50% formaldehyde followed by washing at 65° C. in 2×SSC containing 1% SDS.

In addition, the present invention relates to a vector containing the aforementioned DNA, and more particularly, to an expression vector. The expression vector contains a control sequence such as a promoter or terminator. An ordinary control sequence such as a promoter corresponding to the host used can be used for the control sequence. Prokaryotic cells such as Escherichia coli, eukaryotic microorganisms such as yeasts or filamentous fungi, or higher eukaryotic cells, namely cultured animal cells or cultured plant cells, can be used.

Although a sugar has been reported to first transfer to the 5 position of anthocyanidin during synthesis of rose anthocyanin, as a result of extensive research, an enzyme that transfers sugar to the 3 position has been unexpectedly demonstrated to be present also in roses. In addition, a gene that encodes that enzyme was able to be acquired. Synthesis of anthocyanin can be promoted by expressing this gene in roses or different species of plants. In addition, synthesis of anthocyanin can be inhibited by inhibiting expression thereof. In addition, by inhibiting synthesis of anthocyanin in roses, accumulation of anthocyanidin, and particularly cyanidin, can be promoted, thereby making it possible to also promote accumulation of rosacyanin.

In addition, a gene encoding an enzyme that transfers a sugar to the 3 position of anthocyanidin was clearly demonstrated to be involved in color changes following blooming of rose flowers. On the basis of the expression profile of this gene, the promoter region of this gene is thought to have the function of specifically expressing the gene after blooming. Roses in which flower color changes after blooming can be produced by molecular breeding using this type of anthocyanin synthesis gene involved in changes in flower color after blooming (containing an expression regulatory region such as a promoter of that gene).

### EXAMPLES

The following provides a detailed description of the present invention according to the examples indicated below. Unless specifically stated otherwise, molecular biology techniques were performed in accordance with Molecular Cloning (Cold Spring Harbor Laboratory Press, 2001) and Plant Physiol. (2003) 132, 1652-1663.

### Example 1

### Construction of Rose cDNA Library and Acquisition of Other 3GT cDNA Candidates

An enzyme (A3GT) gene that transfers a sugar to the 3 position of anthocyanidin contained in the petals of plants can be obtained by screening a cDNA library thereof with A3GT genes of different species. For example, gentian A3GT gene was able to be obtained by using snapdragon A3GT gene as a probe (Plant Cell Physiol. (1996) 37, 711-716). A cDNA library was constructed using RNA extracted from colored leaves (in which anthocyanin was accumulated) of roses (Rosa hybrida cultivar Samba) according to a known method (for example, Plant Cell Physiol. (1995) 36, 1023-1031).

Library construction was carried out using the λZAPII Directional cDNA Library Synthesis Kit (Stratagene Corp.) in accordance with the recommended method by the manufacturer. A cDNA library consisting of about 5,500,000 plaque forming units (pfu) was obtained. Portions corresponding to the sequence of the N-terminal sequences of two types of genes consisting of perilla-derived A3GT gene (Plant Mol. Biol. (1997) 35, 915-927) and gentian-derived A3GT gene (Plant Cell Physiol. (1996) 37, 711-716) were used as probes for library screening. A pair of oligonucleotide primers each as indicated below were synthesized to amplify the N-terminal sequence of each GT.

Perilla A3GT
SEQ ID NO. 5: 5′-CAC ATT GGC GTT CTA GCA TTC-3′
SEQ ID NO. 6: 5′-GCC TTC GGG GGT CCC GTC CCA-3′
Gentian A3GT
SEQ ID NO. 7: 5′-CAT GTT GCA GTG CTT GCA TTT-3′
SEQ ID NO. 8: 5′-TCC CTC CGG CGA GCC ATC CCA-3′

The probes were labeled by PCR using the Non-Radioisotope DIG-Nucleic Acid Detection System (Roche Diagnostics K.K.) in accordance with the conditions recommended by the manufacturer. At that time, 1 ng of plasmid containing each cDNA were used as templates, 100 ng of oligonucleotide specific to each gene as described above were used as primers, and PCR was carried out for 35 cycles with each cycle consisting of reacting for 1 minute at 94° C., 1 minute at 55° C. and 1 minute at 72° C. The aforementioned cDNA library (approx. 300,000 pfu for each probe) was screened by using the PCR amplification products of each gene as hybridization probes.

The screening was carried out using the Non-Radioisotope DIG-Nucleic Acid Detection System (Roche Diagnostics K.K.) according to the method recommended by the manufacturer. Hybridization was carried out overnight at 37° C. in 5×SSC containing 30% formaldehyde followed by washing the filter for 30 minutes at 55° C. using 5×SSC containing 1% SDS. Secondary screening was further carried out on those clones for which a signal was detected in primary screening, and ultimately one kind of cDNA (pSPB1123) was obtained.

The resulting nucleotide sequence is shown in SEQ ID NO. 1, and the corresponding amino acid sequence is shown in SEQ ID NO. 2. When this amino acid sequence was compared with a known 3GT amino acid sequence, the amino acid sequence of the enzyme of the present invention demonstrated sequence identity of 42% with respect to perilla A3GT, sequence identity of 43% with respect to gentian A3GT and sequence identity of 44% with respect to grape A3GT (J. Biol. Chem. (1998) 273, 9224-9233). On the other hand, homology with a previously reported rose anthocyanidin 5,3-glucosyltransferase was 22%. The enzyme of the present invention is therefore a novel enzyme on the basis of the above.

### Example 2

### Activity of Protein Encoded by Rose 3GT cDNA Candidate

In order to analyze the activity of a protein encoded by cDNA contained in pSPB1123, this cDNA was expressed in yeast cells. Yeast expression vector pYE22m (Plant Cell Physiol. (1996) 37, 711-716) was used to express the cDNA in yeast. A fragment obtained by digested pSPB1123 cDNA with EcoRI and KpnI was inserted into an EcoRI and KpnI recognition sequence of pYE22m to obtain a yeast expression vector. This vector was then inserted into yeast strain G1315 followed by measurement of activity of the protein encoded by pSPB1123 according to the method described on pp. 114-118 of Shinseibutsugaku Jikken (New Biological Experiment) Manual 3 (Kagaku-Dojin Publishing Co., Inc.).

The method for measuring activity and the conditions for measuring a product by HPLC were the same as the method and the like described in Plant Physiology (2003) 132, 1652-1663. Furthermore, anthocyanin (cyanidin, pelargonidin), cyanidin 3-glucoside (cyanidin 3G), cyanidin 3,5-diglucoside (cyanidin 3,5G), flavonol (quercetin, kaempferol) and flavone (apigenin, luteolin) were used as substrates for measurement of the activity.

As a result, protein encoded by cDNA contained in pSPB1123 had significant activity that added glucose to flavonol, and was observed to tend to prefer quercetin to kaempferol. The product was confirmed to be a 3-glycosylation product of flavonol based on cochromatography with an authentic sample. Although there also appeared to be slight activity to add a glucose to cyanidin as well, when compared with the case of using flavonol as a substrate, only a very small amount of cyanidin 3-glucoside was formed. In addition, glycosylation activity to flavones was not observed. Thus, the glucosyltransferase encoded by this cDNA was determined to be a flavonol 3GT rather than an anthocyanidin 3GT. In addition, even when screening was repeated on the same library using cDNA contained in this pSPB1123 as a probe, a gene of an enzyme that transfers a sugar to the 3 position of anthocyanidin was unable to be obtained, and rose A3GT was unable to be easily obtained.

### Example 3

### Construction of Rose Genomic Library

Genomic DNA was extracted from the petals of a rose (cultivar: Lavande) using Phytopure (Amersham plc), and a genomic library was constructed as indicated below using λBlueSTAR in accordance with the recommended method of the manufacturer.

First, after partially digesting the extracted genomic DNA with a restriction enzyme Sau3AI, a partial fill-in reaction was carried out in accordance with the method recommended by Novagene Inc. followed by fractionating by a sucrose density gradient centrifugation. DNA fragments ranging from 12 to 24 kb were recovered, and after ligating with λBlueSTAR vector digested in advance with XhoI, packaging was carried out using the Gigapack Packaging Kit (Stratagene Corp.). A genomic library consisting of about 1,000,000 pfu was obtained.

### Example 4

### Screening of Rose A3GT Gene

A N-terminal sequence of the GTs disclosed previously, which is highly conserved only in A3GTs were amplified and the rose cDNA library described in Example 1 was screened by using the fragment as probes. The GTs used for the probes consisted of the same perilla A3GT gene (Plant Mol. Biol. (1997) 35, 915-927) and gentian A3GT gene (Plant Cell Physiol. (1996) 37, 711-716) of Example 1 as well as snapdragon A3GT gene (Plant J. (1991) 1, 37) and petunia A3GT (Plant Mol. Biol. (2002) 48, 401), for a total of four types. The perilla and gentian A3GT genes were the same as in Example, while the following respective pairs of oligonucleotides were synthesized for the snapdragon and petunia A3GT genes in order to amplify the DNA sequences corresponding to the N-terminal sequences of each GT.

Snapdragon A3GT
SEQ ID NO. 9: 5′-CAC ATT GGC GTG CTA GCG TTC-3′
SEQ ID NO. 10: 5′-GCC CTC TGG GGT GCC ATC CCA-3′
Petunia A3GT
SEQ ID NO. 11: 5′-CAC ATT GCA CTT CTT GCT TTC-3′
SEQ ID NO. 12: 5′-TGT TTC AGT GAC ACC ATC CCA-3′

A labeled PCR amplification product of each gene prepared in the same manner as Example 1 was used as a hybridization probe to screen the rose genomic library described in Example 3 (approx. 300,000 pfu for each probe).

Screening was carried out using the Non-Radioisotope DIG-Nucleic Acid Detection System (Roche Diagnostics K.K.) according to the method recommended by the manufacturer. Hybridization was carried out overnight at 37° C. in 5×SSC containing 30% formaldehyde followed by washing the filter for 30 minutes at 55° C. using 5×SSC containing 1% SDS. Secondary screening was further carried out for those clones for which a signal was detected in primary screening, and ultimately 14 positive clones were obtained from those clones in which gentian A3GT gene was used as a probe. There were no positive clones obtained in the case of using any of the other A3GT genes than gentian's.

### Example 5

### Analysis of Obtained A3GT Candidate Genes

Plasmid DNA were recovered from the positive clones acquired in Example 2 using the λBlueSTAR Library Synthesis Kit in accordance with the method recommended by the manufacturer. In order to determine which parts of the cloned sequence 3GT gene is present, the obtained plasmid DNA was digested with restriction enzymes XhoI, SalI, HindIII and XbaI (sigle digestion) or using both EcoRI and BamHI followed by carrying out southern hybridization using the same probes derived from gentian A3GT gene used in Examples 1 and 4.

Southern hybridization was carried out by electrophoresis of each restriction enzyme-digested DNA in 8% agarose gel followed by using the aforementioned Non-Radioisotope DIG-Nucleic Acid Detection System DNA Labeling and Detection Kit in accordance with the method recommended by the manufacturer. Those fragments of the resulting genomic clones that demonstrated a strong signal in a southern hybridization were sub-cloned to a pBluescript II SK vector followed by analysis of their nucleotide sequences. Sequencing was carried out using the DNA Sequencer Model 3100 (Applied Biosystems Inc.) according to the primer walking method using synthesized oligonucleotide primers.

As a result of comparing the amino acid sequence predicted from the resulting sequence with those of known A3GT of other plants, a sequence demonstrating high identity with A3GT of other plants was determined to be present in an approximately 3.5 kg fragment resulting from cleavage of clone #16 with HindIII (designated as pSFL333). Identity between the amino acid sequence of the GT present in the sequence of this pSFL333 and the amino acid sequences of other A3GT was 46% with respect to gentian A3GT, 46% with respect to perilla A3GT, and 57% with respect to grape A3GT. In addition, identity at the amino acid level with flavonol 3GT encoded by pSFL1123 described in Example 1 was 42%. On the other hand, identity with previously reported rose anthocyanidin 5,3-glucosyltransferase was 21%.

The entire sequence of the pSFL333 cDNA is shown in SEQ ID NO. 3, while the corresponding amino acid sequence is shown in SEQ ID NO. 4. In addition, an intron was also determined to be present in this rose 3GT candidate gene at the location corresponding to the intron of Arabidopsis thaliana A3GT (UGT78D1). Although there were other genomic clones obtained from screening that had a sequence considered to be a GT gene besides clone #16, since they did not demonstrate high homology with known A3GT, subsequent analysis was focused only on pSFL333 cDNA.

### Example 6

### Construction of Escherichia coli Expression Construct of Resulting Anthocyanin 3GT Candidate Gene

Since an intron of about 180 by was present in the resulted rose 3GT candidate gene about 500 by downstream from the initiating methionine codon as shown in SEQ ID NO. 3, it was necessary to remove this intron when analyzing activity by expressing in E. coli.

Therefore, three fragments were prepared, consisting of (1) a fragment in which the region from the initiating methionine codon to just before the intron was amplified by PCR using Rose 3GT16-NcoI primer and Rose 3GT16-SpeI-1 primer, (2) a fragment in which the region from immediately after the intron to a KpnI recognition sequence present within the second exon was amplified by PCR using Rose 3GT-SpeI-3 primer and Rose 3GT-KpnI primer, and (3) a fragment in which the region from a KpnI recognition sequence present within the second exon to a BamHI recognition sequence present within the 3′ untranslated region, and fragments (2) and (3) followed by fragment (1) were inserted into the NcoI and BgIII recognition sequences of E. coli expression vector pQE61 (Qiagen, Inc.) in that order to construct an E. coli expression vector.

Furthermore, the nucleotide sequences of fragments (1) and (2) amplified by PCR were confirmed, and digested with NcoI and SpeI or SpeI and KpnI prior to insertion. The resulting E. coli expression vector was designated as pSFL344. The following indicates the sequences of the primers used to amplify fragments (1) and (2).

SEQ ID NO. 13: Rose 3GT16-NcoI
5′-GAC TCC ATG GCA CCA GCA TCA AAT C-3′
SEQ ID NO. 14: Rose 3GT16-SpeI-1
5′-ACA CGT ACT AGT TGT GTT CCT GAT G-3′
SEQ ID NO. 15: Rose 3GT16-SpeI-3
5′-AGT CAC TAG TAT GGG TGG TCA CGA CGA TG-3′
SEQ ID NO. 16: Rose 3GT16-KpnI
5′-CGT TGA GGT ACC TCT TGA ATT TGG-3′

### Example 7

### Measurement of Activity of Rose Anthocyanin 3GT

Expression constructs pSFL344 and pQE61 were introduced into E. coli strain JM109 (Toyobo Co., Ltd.) and pre-cultured overnight at 37° C. in LB medium containing ampicillin at a final concentration of 20 μg/ml. A portion of the pre-culture broth was cultured to an A600 of 0.6 to 1.0 for about 2 hours in M9 medium containing 50 μg/ml of ampicillin and 0.5% casamino acids, brought to a volume of 10 ml, followed by the addition of IPTG (isopropyl-β-D-thiogalactopyranoside) to a final concentration of 0.1 mM and further culturing with shaking for 4 hours at 37° C. After centrifuging for 10 minutes at 3000 rpm and 4° C., the collected cells were suspended in 10 ml of buffer (30 mM Tris-HCl pH 7.5, 30 mM NaCl) followed by lysing the E. coli by ultrasonic treatment, carrying out centrifugal precipitation for 10 minutes at 15000 rpm and 4° C. and using the resultant supernatant as a crude enzyme liquid for use in the following measurement of enzyme activity.

The reaction was carried out by mixing 30 μl of the crude enzyme liquid, 10 μl of 1 M Tris-HCl (pH 7.5), 5 μl of 5 mM UDP-glucose and 5 μl of 2 mM delphinidin or cyanidin, followed by holding for 15 minutes at 30° C. After stopping the reaction by adding an equal amount of 90% acetonitrile containing 0.1% TFA, the glycosylated products were detected in the same manner as the method described in Plant Physiol. (2003) 132, 1652-1663. In the reaction system using crude enzyme derived from E. coli expressing pSFL344, a specific product was detected that was not observed in the reaction system using crude enzyme derived from E. coli into which pQE60 is introduced. This product was identified as delphinidin 3-glucoside or cyanidin 3-glucoside based on cochromatography with a standard and agreement with the absorption spectrum of the standard.

On the basis of these results, a gene encoded by pSFL333 was confirmed to be that which encodes A3GT having activity that adds a sugar to the 3 position of anthocyanin.

### Example 8

### Inhibition of Expression of Flavonol 3GT Encoded by pSBP1123 cDNA in Roses

In order to assess effects in the case of inhibiting expression of the previously described rose anthocyanin 3GT (A3GT) and flavonol 3GT (F3GT) in rose petals, constructs were prepared for inhibiting expression of each 3GT and simultaneously inhibiting expression of both 3GT. First, with respect to flavonol 3GT encoded by pSPB1123, (1) a fragment extending from an Sad recognition sequence originating in the 5′-side vector to an SphI recognition sequence present in the vicinity of 900 by from the 5′-terminal of the cDNA, and (2) a fragment extending from a BamHI recognition sequence originating in 5′-side vector sequence to an SphI recognition sequence inserted by PCR at a location about 750 by from the 5′-terminal of the cDNA, were prepared.

The fragment of (2) was prepared by digesting with BamHI and SphI a DNA fragment amplified by PCR using the following primers by an ordinary methods (pSPB1123-SphI primer and reverse primer originating in the vector sequence).

SEQ ID NO. 17: pSPB1123-SphI
5′-AGA GTT GCA TGC AAC CGC AGT T-3′
SEQ ID NO. 18: Reverse primer
5′-AAC AGC TAT GAC CAT G-3′

A GUS gene located between an EI₂35S promoter and mas terminator of binary expression vector pSPB528 in the plant described in International Publication WO 2005/017147 was removed by cleaving with BamHI and SacI, a total of three DNA fragments consisting of this DNA fragment and the DNA fragments of (1) and (2) above were ligated to obtain pSFL224. Plasmid pSFL224 obtained in this manner, when introduced into a rose, constitutively transcribes double-stranded RNA originating in flavonol 3GT cDNA which pSPB1123 has, under the control of E1235S promoter. As a result, expression of rose flavonol 3GT is expected to be inhibited by RNAi.

### Example 9

### Inhibition of Expression of A3GT Encoded by pSFL333 cDNA in Roses

Anthocyanin 3GT encoded by pSFL333 was prepared by respectively digesting fragments consisting of (1) a fragment extending from an XhoI recognition sequence present in the vicinity of 50 by from the 5′-terminal to an SpeI recognition sequence present in the vicinity of 835 by from the 5′-terminal, and (2) a fragment extending from a BamHI recognition sequence originating in the 5′-side vector sequence to an SpeI recognition sequence present in the vicinity of about 630 by from the 5′-terminal of the cDNA, with restriction enzymes, followed by electrophoresing and recovering the fragments from the gel. During preparation of fragment (1), SpeI-digestion was carried out by partial digestion by limiting the digestion time.

On the other hand, a plasmid in which a fragment extending from an HindIII recognition sequence to an EcoRI recognition sequence cut out from the vector pUE8 described in International Publication WO 2005/017147 (extending from an EI₂35S promoter to an nos terminator), is inserted to a HindIII recognition sequence and an EcoRI recognition sequence of pBINPLUS (Van Engelen et al., Transgenic Research 4 (1995) 288-290), was designated as pSPB176. A total of three fragments, consisting of a DNA fragment from which GUS gene had been removed by digesting with BamHI and SalI, and fragments (1) and (2) above, were then ligated to obtain pSFL223. The resulting plasmid pSFL223, when inserted into a rose, constitutively transcribes double-stranded RNA originating in A3GT cDNA contained in pSFL333 under the control of EI₂35S promoter. As a result, expression of rose A3GT is expected to be inhibited by RNAi.

### Example 10

### Inhibition of Expression of Both 3GT Encoded by pSPB1123 and pSFL333 cDNA in Roses

A construct was prepared for inhibiting both 3GT activities encoded by pSFL1123 and pSFL333. After digesting vector pSPB313 described in International Publication WO 2005/059141 with BamHI and Sad and removing F3H cDNA, this DNA fragment was ligated to fragments (1) and (2) described in Example 9, and the resultant plasmid was designated as pSFL225. A fragment extending from a promoter to a terminator obtained by cleaving pSFL225 with AscI was inserted into the AscI recognition sequence of pSFL223 prepared in Example 9 so as to have the same transcription orientation as the expression cassette of pSFL223, and the resulting construct was designated as pSFL226.

This pSFL226, when introduced into a rose, transcribes double-stranded RNA originating in pSFL1123 and pSFL333 cDNA under the control of EI₂35S promoter. As a result, expression of both of these rose flavonol and anthocyanidin 3GT is expected to be inhibited by RNAi.

### Example 11

### Analysis of Expression Mode of F3GT Gene Encoded by pSPB1123 in Roses

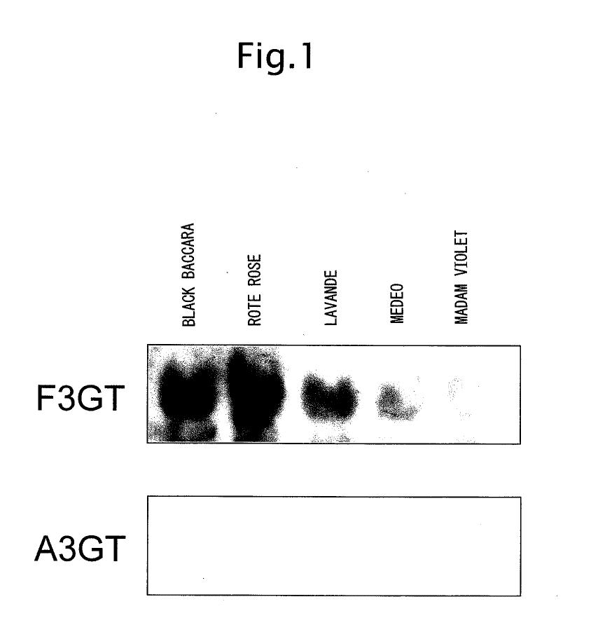

In order to analyze the expression mode of F3GT gene encoded by pSBP1123 in roses, RNA was extracted from the petals of rose cultivars consisting of Black Baccara, Rote Rose, Medeo, Madam Violet and Lavande during coloring (Plant Cell Physiol. (1995) 36, 1023-1031), followed by Northern hybridization in accordance with a known method (Plant Cell Physiol. (1995) 36, 1023-1031). Northern hybridization was carried out using a DIG Northern Kit (Amersham plc) according to the method recommended by the manufacturer. An RNA probe originating in the F3GT gene sequence labeled with DIG was used for the probe. Transcription products of the expected lengths were detected for all cultivars (FIG. 1).

### Example 12

### Analysis of Expression Mode of A3GT Gene Encoded by pSFL333 in Roses

In order to analyze the expression mode of A3GT gene encoded by pSFL333 in roses, RNA was extracted from the petals of roses followed by Northern hybridization. Northern hybridization was carried out using an RNA probe originating from an A3GT gene sequence labeled with DIG in the same manner as Example 11.

In rose cultivars such as Black Baccara, Rote Rose, Lavande, Medeo or Madam Violet, hardly any transcription products were detected even at the bud stage during which structural genes involved in anthocyanin synthesis in colored petals are normally expressed at a high level (Plant Cell Physiol. (1995) 36, 1023-1031) (FIG. 1). When the anthocyanin present in these cultivars of roses is analyzed, although cyanidin 3G is normally contained in extremely small amounts in petals immediately after blooming from buds, cyanidin 3,5G is present as nearly a single product.

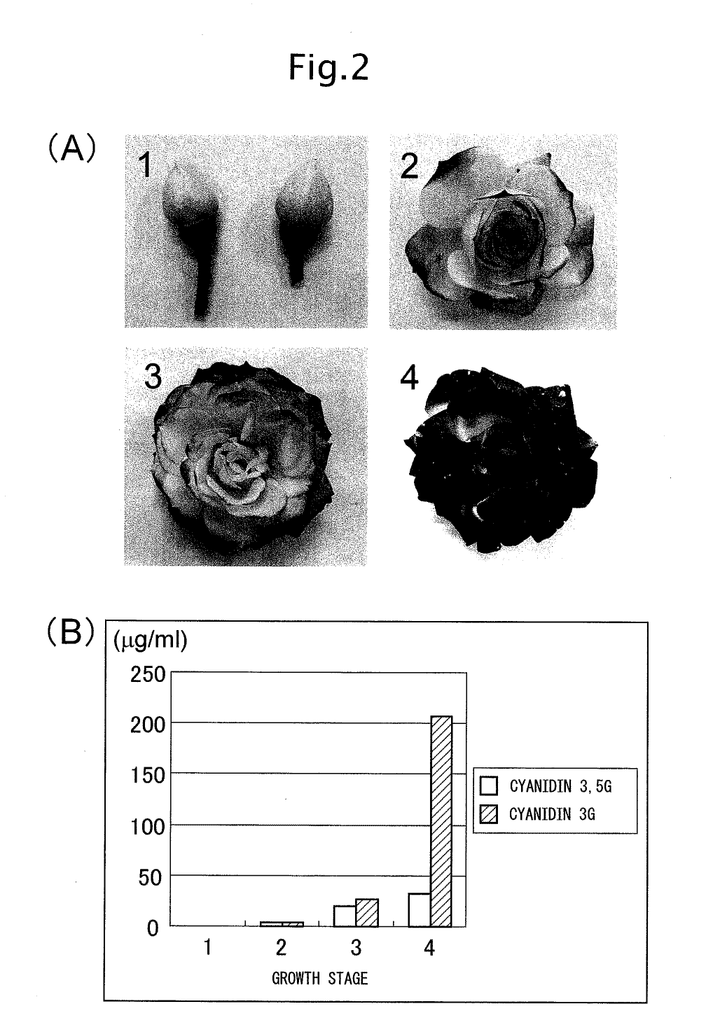

On the other hand, in rose cultivar such as Ehigasa (Keihan Gardening Co., Ltd.) or Charleston that change color accompanying blooming, although similar to cultivar mentioned above, cyanidin 3,5G is present in large amounts in petals that are initially orange-yellow, accompanying subsequent blooming, cyanidin 3G has been demonstrated to be accumulated in large amounts at the stage when the flowers become red (FIG. 2).

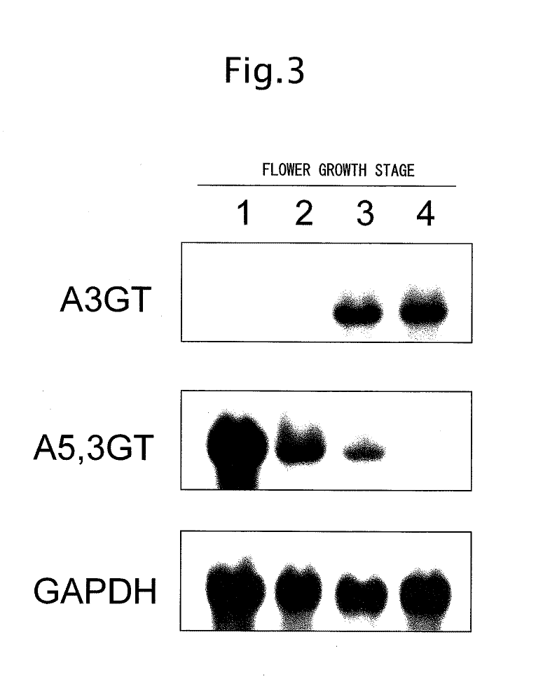

Therefore, RNA was extracted from the petals of a rose such as Ehigasa, which changes color accompanying blooming in this manner, from the stage of young bud to the stage at which aging starts after blooming (FIG. 2), and Northern hybridization was carried out using an RNA probe originating in A3GT gene sequence encoded by pSFL333 in the manner described above. As a result, this A3GT was determined be prominently expressed from the stage at which color change begins after blooming to the stage at which red coloring progresses, or in other words, during the stage at which cyanidin 3G accumulates (FIG. 3).

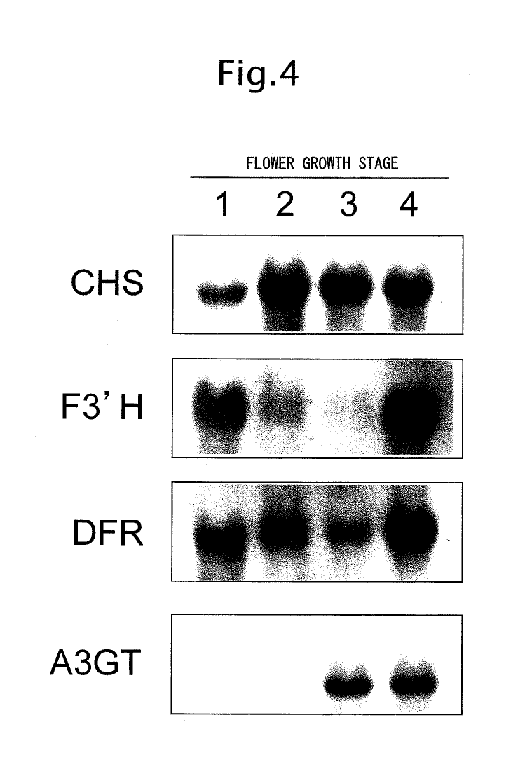

In addition, as a result of carrying out Northern hybridization using RNA probes originating in the respective gene sequences of rose chalcone synthase (CHS), dihydroflavonol reductase (DFR) and flavonoid 3′-hydroxylase (F3′H) that act as structural genes involved in the synthesis of anthocyanin in the same manner as described above, these genes were also determined to be prominently expressed during stage 4 in which this A3GT is expressed (FIG. 4). Each of these CHS, DFR and F3′H genes were expressed at higher levels in stage 4, during which cyanidin 3G is accumulated and petals become nearly completely red, rather than in stage 1, during which A5,3GT gene is expressed at a high level. On the basis of this finding, in cultivars such as Ehigasa that change cover after blooming, it is believed that the entire anthocyanin synthesis system is activated in the stage after blooming when clear aging begins (stage 4) rather than the stage during which anthocyanin synthesis is most active in ordinary roses (stage 1).

Moreover, on the basis of the above results, promoters and other transcription regulatory regions of this A3GT gene are predicted to have a function that specifically expresses this gene in petals that have progressed to the stage after blooming. Accordingly, it is thought to be possible to create a rose that changes color after blooming by ligating a coding region of a pigment synthesis gene to a promoter portion of A3GT shown in SEQ ID NO. 3 and inserting into a suitable rose. For example, if cDNA of pansy F3′,5′H (International Publication WO 2004/020637) is ligated to the sequence of a promoter portion of A3GT and inserted into a suitable cultivar of rose, then it is thought that blue delphinidin will be synthesized accompanying blooming, making it possible to create a rose that changes to a blue color.

### Example 13

### Analysis of Rose Transformants Inserted with Construct Inhibiting F3GT Encoded by pSBP1123

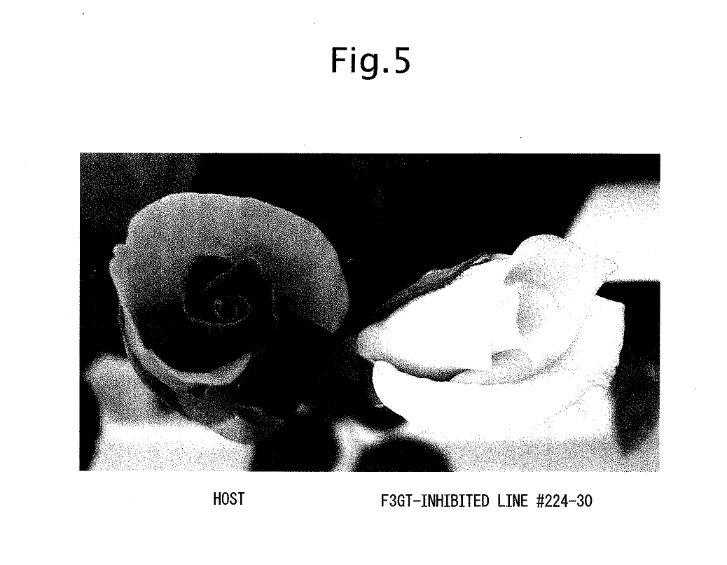

pSFL224 was inserted into rose cultivar Lavande to suppress F3GT encoded by pSPB1123 as in Example 8. The flower colors of several transgenic lines became paler or whiter as compared with the host (FIG. 5). As a result of analyzing the flavonoid contents of the flowers of those lines that exhibited color changes in this manner by HPLC, the content of cyanidin 3,5G, which accounted for nearly 100% of the anthocyanin contained by the host petals, had decreased to roughly one-sixth that level in the transformants. In addition, flavonol content had increased to roughly twice that of the control. In addition, rosacyanin content remained unchanged.

DNA was extracted from the petals of the transformants that exhibited a change in petal color to confirm insertion of the construct down-regulating F3GT gene in the form of pSFL224.

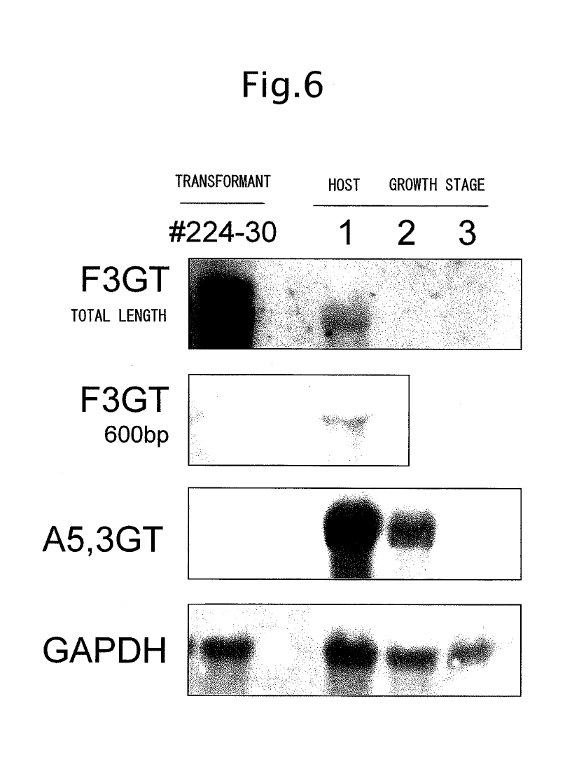

Moreover, RNA was extracted from transformant #224-30, in which a change in petal color was observed, and from petals of the host Lavande cultivar, and Northern hybridization was carried out using RNA probes respectively originating in sequences consisting of the total length of F3GT gene, a 600 by fragment on the 3′-side of F3GT gene, and rose A5,3GT gene (FIG. 6). In the case of using the full length of F3GT gene for the probe, the F3GT transcription product was higher in the transformants than in the host (FIG. 6). However, in the case of using a portion of endogenous F3GT gene that is not contained in pSFL224, namely a portion specific to endogenous F3GT gene, for the probe, although the transcription product was detected in the host, there were transcription products detected in the transformants. Here, the portion specific to endogenous F3GT gene used for the probe refers to a fragment which is not contained in pSFL224 and has about 600 by on the 3′-terminal side from an SphI recognition sequence located at a location about 900 by from the methionine starting codon of F3GT gene. Thus, although expression of endogenous F3GT gene is inhibited in transformants inserted with pSFL224, transcription products originating in the F3GT sequence of the inserted pSFL224 are thought to be present at a high level. In addition, transcription products were detected in the transformants that were of a different length than the inherently transcribed sequence.

On the other hand, the transcription product of A5,3GT was significantly reduced in transformants in which petal color became lighter. Expression of rose GAPDH gene, which was used as a positive control, was constant in all lines (FIG. 6).

On the basis of these results, in addition to expression of endogenous F3GT gene being suppressed by insertion of pSFL224 construct that inhibits expression of F3GT gene, expression of endogenous A5,3GT gene, which is said to be required for formation of anthocyanin, was also inhibited. As a result, the amount of anthocyanin in the pelals of the transformants decreased, which was thought to cause color to become lighter. In the case of inhibiting expression of a target gene by inserting an RNAi construct, although the transcription product of the RNAi construct itself typically decreases or disappears in coordination with the transcription product of the target gene, in this case, F3GT gene originating in the inserted pSFL224 is thought to have been expressed in excess even though expression of the target gene (rose endogenous F3GT gene) was inhibited.

### Example 14

### Analysis of Rose Transformants Inserted with Construct Inhibiting Expression of A3GT Encoded by pSFL333

pSFL223 was introduced into rose cultivar Lavande to inhibit expression of A3GT encoded by pSFL333 as in Example 9. Although about 30 transformants were obtained, none were observed to exhibit changes in flower color. In addition, although the pigments of five of the transformants were analyzed by HPLC, there were no differences between the contents of anthocyanin and rosacyanin contained in the petals immediately after blooming and the contents in the control.

In addition, as was described in Example 12, A3GT encoded by pSFL333 is virtually not expressed or only expressed at a low level at which transcription products are unable to be confirmed by Northern hybridization in the host Lavande rose (FIG. 1). Accordingly, although the presence or absence of transcription products of A3GT gene in the transformants was analyzed by Northern hybridization, no transcription products were detected in any of the transformants in the same manner as the control.

### INDUSTRIAL APPLICABILITY

As indicated in Example 13, rose F3GT gene encoded by pSFL1123 inhibits anthocyanin synthesis in roses, and can be used to produce roses that change color.

On the other hand, A3GT gene encoded by pSFL333 was determined to be specifically involved in anthocyanin synthesis in the stage in which color changes following blooming, instead of anthocyanin synthesis in the stage of the initial bud, in rose cultivars that change color such as Picture Parasol. On the basis of this finding, this A3GT gene, and particularly a promoter region thereof, can be used to produce flowers that change color accompanying blooming by genetic recombination.

1. A nucleic acid encoding a protein originating in Rosaceae Rosa having activity that transfers a sugar to a 3-position hydroxyl group of a flavonoid.

2. The nucleic acid according to claim 1, wherein the flavonoid is anthocyanidin or flavonol.

3. A nucleic acid encoding a protein having an amino sequence indicated in SEQ ID NO. 2 or SEQ ID NO. 4, which has activity that transfers a sugar to a 3-position hydroxyl group of a flavonoid.

4. A nucleic acid encoding a protein having an amino acid sequence that has been modified by addition, deletion and/or substitution with other amino acids to one or a plurality of amino acids in an amino acid sequence indicated in SEQ ID NO. 2 or SEQ ID NO. 4, and having activity that transfers a sugar to a 3-position hydroxyl group of a flavonoid.

5. A nucleic acid encoding a protein having an amino acid sequence that has been modified by addition, deletion and/or substitution with other amino acids to one or several amino acids in an amino acid sequence indicated in SEQ ID NO. 2 or SEQ ID NO. 4, and having activity that transfers a sugar to a 3-position hydroxyl group of a flavonoid.

6. A nucleic acid encoding a protein having sequence identity of at least 60% with respect to an amino acid sequence indicated in SEQ ID NO. 2 or SEQ ID NO. 4, and having activity that transfers a sugar to a 3-position hydroxyl group of a flavonoid.

7. A nucleic acid encoding a protein having sequence identity of at least 80% with respect to an amino acid sequence indicated in SEQ ID NO. 2 or SEQ ID NO. 4, and having activity that transfers a sugar to a 3-position hydroxyl group of a flavonoid.

8. A nucleic acid encoding a protein having sequence identity of at least 90% with respect to an amino acid sequence indicated in SEQ ID NO. 2 or SEQ ID NO. 4, and having activity that transfers a sugar to a 3-position hydroxyl group of a flavonoid.

9. A nucleic acid encoding a protein having an amino acid sequence encoded by a nucleic acid that hybridizes under moderately stringent conditions with a nucleic acid having a nucleotide sequence of a coding region indicated in SEQ ID NO. 1 or SEQ ID NO. 3, and having activity that transfers a sugar to a 3-position hydroxyl group of a flavonoid.

10. A nucleic acid encoding a protein having an amino acid sequence encoded by a nucleic acid that hybridizes under high stringent conditions with a nucleic acid having a base sequence of a coding region indicated in SEQ ID NO. 1 or SEQ ID NO. 3, and having activity that transfers a sugar to a 3-position hydroxyl group of a flavonoid.

11. A nucleic acid according to claim 3, containing a nucleotide sequence of a coding region indicated in SEQ ID NO. 1 or SEQ ID NO. 3.

12. A nucleic acid containing a sequence complementary to the nucleic acid according to claim 1.

13. The nucleic acid according to claim 1, wherein the nucleic acid is DNA or RNA.

14. A vector containing the nucleic acid according to claim 1.

15. A host transformed by the vector according to claim 14.

16. A protein encoded by the nucleic acid according to claim 1.

17. A method of producing a protein having activity that transfers a sugar to a 3-position hydroxyl group of a flavonoid, comprising: culturing or growing the host according to claim 15, and harvesting the protein from the host.

18. A plant into which the nucleic acid according to claim 1 has been inserted, a progeny thereof having identical properties thereto, or tissue or vegetative growth body thereof.

19. A plant into which the nucleic acid according to claim 1 has been inserted and for which flower color has been altered, progeny thereof having identical properties thereto, or tissue or vegetative growth body thereof.

20. A plant according to claim 18, or a cut flower of a progeny thereof having identical properties thereto.

21. A method for producing a plant in which color changes as a result of blooming comprising the use of the gene described in SEQ ID NO. 3 or a portion thereof.

22. The method according to claim 21, wherein the plant referred to in claim 21 is a rose.

23. A plant that changes color after blooming produced according to the method according to claim 21, progeny thereof having identical properties thereto, or tissue or vegetative growth body thereof.

24. The plant according to claim 23, or a cut flower of a progeny thereof having identical properties thereto.
